(19) United States
(12) Patent Application Publication
(10) Pub. No.: US 2024/0148234 A1
(43) Pub. Date: May 9, 2024

(54) LIGHT PROCESSING ADAPTER FOR ENDOSCOPE

(71) Applicant: GYRUS ACMI, INC. D/B/A OLYMPUS SURGICAL TECHNOLOGIES AMERICA, Westborough, MA (US)

(72) Inventors: Jeffery D. Penman, Ogden, UT (US); Robert E. Ailinger, Norwood, MA (US); Eric A. Frattura, Shrewsbury, MA (US)

(21) Appl. No.: 18/488,522

(22) Filed: Oct. 17, 2023

Related U.S. Application Data
(60) Provisional application No. 63/382,644, filed on Nov. 7, 2022.
(60) Provisional application No. 63/486,507, filed on Feb. 23, 2023.

(51) Int. Cl. A61B 1/00; A61B 1/05

(52) U.S. Cl. CPC A61B 1/00006; A61B 1/00018; A61B 1/0002; A61B 1/00059; A61B 1/00096; A61B 1/00124; A61B 1/00186; A61B 1/05

(57) ABSTRACT

An adapter for an endoscope system comprises a housing, a light conducting element, a sensor to receive light waves emitted from the light conducting element, a converter connected to the sensor to convert light waves into an electrical signal comprising instructions for generating light with a light generator of an endoscope, and an electrical coupler connected to the converter to convey the electrical signal out of the housing to the endoscope. A method for communicating light control signals from an imaging and control system to an endoscope having light generating capabilities comprises generating light with a light generator, receiving light from the light generator at an adapter, sensing an intensity of light with a sensor, converting the intensity sensed by the sensor to light control signals for generating light with a light generator of the endoscope, and transmitting the light control signal to the light generator of the endoscope.

### PRIORITY CLAIM

This application claims the benefit of priority to U.S. Provisional Patent Application Ser. No. 63/382,644, filed Nov. 7, 2022, and. U.S. Provisional Patent Application Ser. No. 63/486,507, filed Feb. 23, 2023; the contents of which are hereby incorporated by reference.

### TECHNICAL FIELD

The present disclosure relates generally to medical devices comprising elongate bodies configured to be inserted into incisions or openings in anatomy of a patient to provide diagnostic or treatment operations.

More specifically, the present disclosure relates to systems and devices for establishing connectivity between medical devices and imaging and control systems.

### BACKGROUND

Endoscopes can be used for one or more of 1) providing passage of other devices, e.g., therapeutic devices or tissue collection devices, toward various anatomical portions, and 2) imaging of such anatomical portions. Such anatomical portions can include the gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting a therapeutic device, such as with the use of an elevator. In some systems, two endoscopes can be configured to work together with a first endoscope guiding a second endoscope inserted therein with the aid of the elevator. Such systems can be helpful in guiding endoscopes to anatomic locations within the body that are difficult to reach. For example, some anatomic locations can only be accessed with an endoscope after insertion through a circuitous path. For example, duodenoscopy procedures (e.g., Endoscopic Retrograde Cholangio-Pancreatography, hereinafter “ERCP” procedures) involve the use of an auxiliary scope (also referred to as a daughter scope or cholangioscope) that can be advanced through the working channel of a main scope (also referred to as a mother scope or duodenoscope). Furthermore, another device, such as a tissue retrieval device used for biopsies, can be inserted into the auxiliary scope. Typically, a duodenoscope, auxiliary scope and tissue retrieval device become progressively smaller since such scopes are configured in telescoping arrangements. Typically, after each use, the duodenoscope, auxiliary scope and tissue retrieval device are cleaned and sterilized for reuse. As such, imaging and control systems have light generators, image processing capabilities and treatment functionality are typically configured for repeated use with the same type or same types of endoscopes and instruments.

### SUMMARY

The present disclosure recognizes that problems to be solved with surgical systems involve the need to adapt disposable endoscopes for use with existing imaging and control systems. There has been a recent desire to utilize disposable endoscopes to, for example, eliminate the need to clean, sterilize and reprocess reusable scopes. However, much capital equipment, such as light generators, image processing equipment and treatment equipment, is configured for use with reusable endoscopes having specific compatibilities, such as lighting and imaging system compatibility. In particular, many endoscopes include light transmitting capabilities, such as light conductors or light pipes, that transmit light generated at the imaging and control system to the distal end of the endoscope for use in the anatomy. As such, the operator of the endoscope can control the imaging and lighting features of the endoscope from the imaging and control system. Thus, there is a need to produce disposable endoscopes that are both compatible with existing imaging and control systems and that are inexpensive.

The present disclosure can provide solutions to these and other problems by providing systems, devices and methods relating to adapters that can transmit lighting instructions from an imaging and control system to an endoscope, particularly a disposable endoscope having an on-board light generator. It can be desirable to produce disposable endoscopes that include a light generator, such as a light emitting diode (LED), instead of a light transmitter. LED light generators can be less expensive than light transmitters, such as light fibers. Furthermore, light fibers can be delicate and subject to fracture if mishandled. However, the removal of the light conductor from the endoscope eliminates the ability of an imaging and control system to control the light output at the distal end of the endoscope. For example, instructions entered into the imaging and control system for the light generator in the imaging and control system will not change the light generated by a light generator in an endoscope since no electronic signal from the light generator of the imaging and control system is communicated to the endoscope. With the present disclosure, an endoscope adapter can be configured to provide lighting instructions to a light generator within an endoscope based on lighting instructions entered into the imaging and control system. In examples, the adapters of the present disclosure can include one or more light sensors that convert light generated by the imaging and control system, and passed into the adapter, into instructions for the light generator in the endoscope. The one or more sensors can sense parameters of light generated at the imaging and control system and convert the sensed parameters into instructions for the light generator in the endoscope to generate light having the same parameters. In a particular example, a light intensity sensor can be used to measure or sense the intensity of light transmitted to the adapter from the imaging and control system and then convert the sensed intensity into electronic instructions for generating light with a light generator in an endoscope. Furthermore, a light color sensor can be used to measure or sense the color of light transmitted to the adapter from the imaging and control system and then convert the sensed color into electronic instructions for generating light with a light generator in the endoscope. As such, existing imaging and control systems, as well as associated operating procedures, can be used with endoscopes having on-board light generators, including disposable endoscopes.

In an example, an adapter for an endoscope system can comprise a housing, a light conducting element extending into the housing, a sensor disposed within the housing to receive light waves emitted from the light conducting element, a converter connected to the sensor to convert light waves into an electrical signal comprising instructions for generating light with a light generator of an endoscope, and an electrical coupler connected to the converter and accessible through the housing configured to convey the electrical signal out of the housing to the endoscope.

In another example, a surgical endoscope system can comprise an imaging and control system comprising a light source having a socket, an endoscope comprising a shaft comprising a coupler at a proximal end portion and an imaging device at a distal end portion, a working channel extending at least partially through the shaft and a light generator configured to emit light proximate the distal end portion, and an adapter configured to be connected to the socket, the adapter comprising a light sensor configured to receive light waves from the light source when the adapter is connected to the socket, a socket configured to receive the coupler of the endoscope and a converter configured to transform light intensity readings from the light sensor into instructions for operating the light generator.

In an additional example, a method for communicating light control signals from an imaging and control system to an endoscope having light generating capabilities can comprise generating light with a first light generator of the imaging and control system, receiving the light from the first light generator at an adapter connected to the imaging and control system, sensing an intensity of the light with a sensor of the adapter, converting the intensity sensed by the sensor to a light control signal for generating light with a second light generator of the endoscope, and transmitting the light control signal to the second light generator of the endoscope through the adapter, wherein the light control signal is configured to instruct the second light generator of the endoscope to generate light of equivalent intensity sensed by the sensor.

### BRIEF DESCRIPTION OF DRAWINGS

FIG. 1 is a schematic diagram of an endoscopy system comprising an imaging and control system and an endoscope, such as duodenoscope, with which the light processing adapters of the present disclosure can be used.

FIG. 2 is a schematic diagram of the endoscopy system of FIG. 1 showing the imaging and control system connected to the endoscope.

FIG. 3 is a block diagram showing a light guide connector coupling the imaging and control system and endoscope of FIGS. 1 and 2.

FIG. 4 is a block diagram showing a light processing adapter of the present disclosure coupling the imaging and control system of FIGS. 1 and 2 to an endoscope having an on-board light generator.

FIG. 5 is a rear perspective view of an adapter of the present disclosure showing connections for coupling to an imaging and control system.

FIG. 6 is a front perspective view of the adapter of FIG. 5 showing a socket for coupling to an endoscope.

FIG. 7 is a rear end view of the adapter of FIGS. 5 and 6 showing a light conductor and an air coupler for connecting to an imaging and control system.

FIG. 8 is a front end view of the adapter of FIGS. 5 and 6 showing an electronics port, an air port and alignment posts for connecting to an endoscope.

FIG. 9 is a front perspective view of the adapter of FIGS. 5-8 with an outer housing removed to show a support bracket connected to a plug component and a socket component.

FIG. 10 is a front perspective view of the adapter of FIG. 9 with the support bracket and socket component removed to show a light conductor, a sensor board, a communications port, an air tube and an air outlet.

FIG. 11 is a cross-sectional view through the adapter of FIG. 10 showing an air passage through the adapter and a light sensor positioned between a light conductor and a communications port.

FIG. 12 is a cross-sectional view through the adapter of FIG. 10 showing a light conductor aimed at a light sensor mounted to a sensor board connected to a communications port.

FIG. 13 is a cross-sectional view through the adapter of FIG. 10 showing a light conductor assembly comprising a light conductor, light filters, an end cap and associated sheathing.

FIG. 14 is a block diagram illustrating an example of a light processing adapter of the present disclosure.

FIG. 15 is a block diagram illustrating operations of methods for converting light generated by an imaging and control system into light control signals for a light generator of an endoscope.

### DETAILED DESCRIPTION

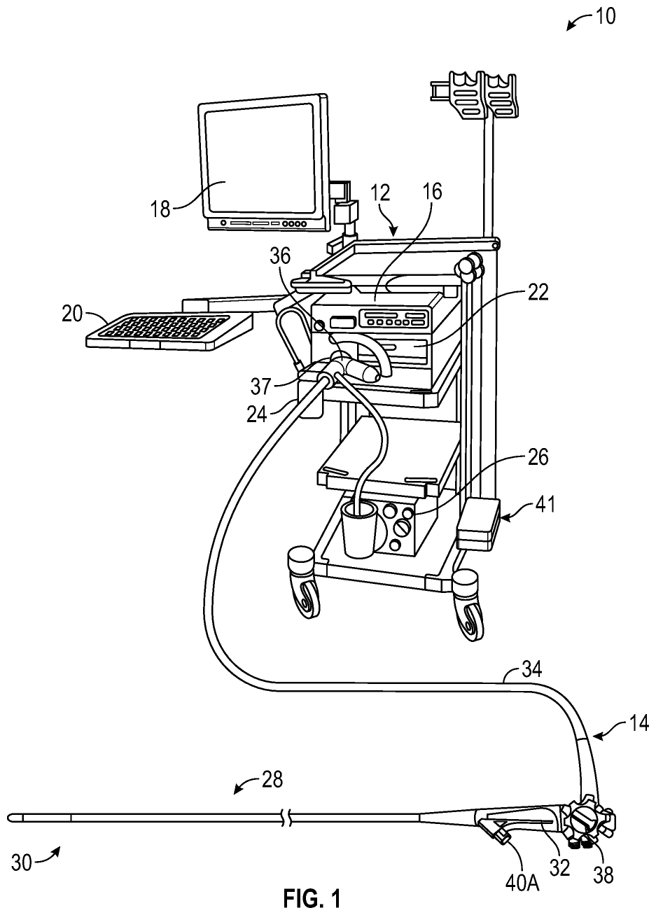

FIG. 1 is a schematic diagram of endoscopy system 10 comprising imaging and control system 12 and endoscope 14. The system of FIG. 1 is an illustrative example of an endoscopy system suitable for use with the systems, devices and methods described herein, such as light processing adapters. According to some examples, endoscope 14 can be insertable into an anatomical region for imaging and/or to provide passage of other devices, such as auxiliary scopes and biopsy devices or one or more therapeutic devices for treatment of a disease state associated with the anatomical region. Endoscope 14 can, in advantageous aspects, interface with and connect to imaging and control system 12 such as via insertion of coupler section 36 into socket 37. In the illustrated example, endoscope 14 comprises a duodenoscope, though other types of endoscopes can be used with the features and teachings of the present disclosure.

Imaging and control system 12 can comprise control unit 16, output unit 18, input unit 20, light source unit 22, fluid source 24 and suction pump 26.

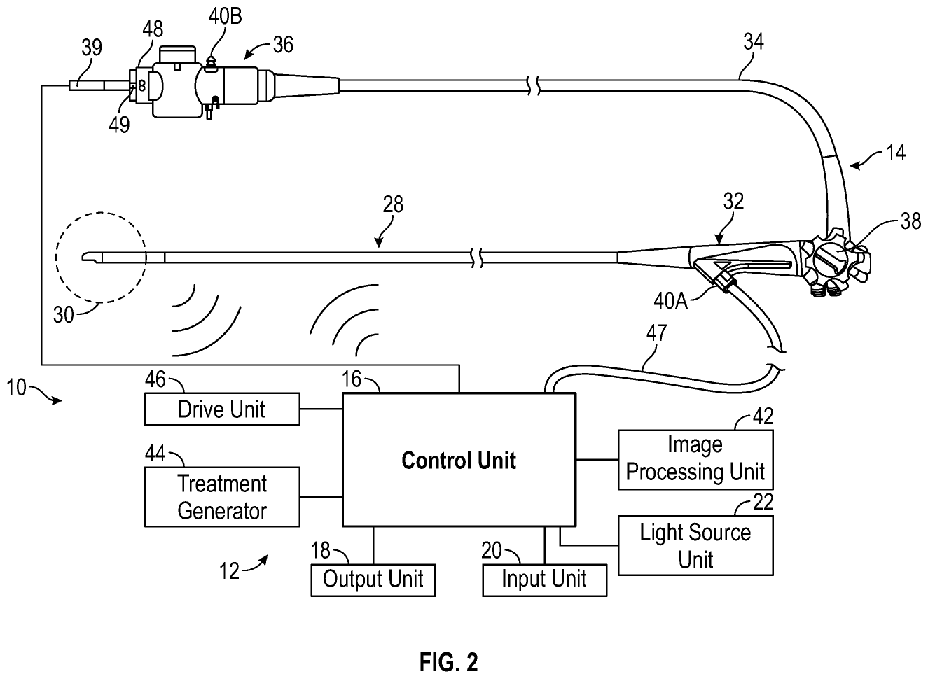

Imaging and control system 12 can include various ports for coupling with endoscopy system 10. For example, control unit 16 can include a data input/output port for receiving data from and communicating data to endoscope 14. Such data input/output can be provided through an interface between coupler section 36 and socket 37. Light source unit 22 can include an output port for transmitting light to endoscope 14, such as via a fiber optic link. For example, coupler section 36 can include light conductor 39 (FIG. 2) that is configured to receive light from a lens or bulb within light source unit 22. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. In examples, fluid, such as air, can be transferred to endoscope 14 through an interface at coupler section 36 and socket 37. In examples, fluids, such as water, can be directly input into coupler section 36 without emanating from socket 37. Output unit 18, e.g., a touch-screen display, and input unit 20, e.g., a keyboard, can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36. Coupler section 36 can be connected to control unit 16 at socket 37 to connect to endoscope 14 to multiple features of control unit 16, such as input unit 20 and light source unit 22. Fluid source 24 and suction pump 26 can be connected directly to endoscope 14 without routing through control unit 16.

Insertion section 28 can extend distally from handle section 32 and cable section 34 can extend proximally from handle section 32. Insertion section 28 can be elongated and include a bending section, and a distal end to which functional section 30 can be attached. The bending section can be controllable (e.g., by control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as an auxiliary scope. The working channel can extend between handle section 32 and functional section 30. Additional functionalities, such as fluid passages, guide wires, and pull wires can also be provided by insertion section 28 (e.g., via suction or irrigation passageways, and the like).

Handle section 32 can comprise control knob 38 as well as port 40A. Control knob 38 can be coupled to a pull wire, or other actuation mechanisms, extending through insertion section 28. Port 40A, as well as other ports, such as port 40B (FIG. 2), can be configured to couple various electrical cables, guide wires, auxiliary scopes, tissue collection devices, fluid tubes and the like to handle section 32 for coupling with insertion section 28.

Imaging and control system 12, according to examples, can be provided on a mobile platform (e.g., cart 41) with shelves for housing light source unit 22, suction pump 26, image processing unit 42 (FIG. 2), etc. Alternatively, several components of imaging and control system 12 shown in FIGS. 1 and 2 can be provided directly on endoscope 14 so as to make the endoscope “self-contained.”

Functional section 30 can comprise components for treating and diagnosing anatomy of a patient. Functional section 30 can comprise an imaging device, an illumination device (e.g., the distal end of a light fiber) and an elevator. Operation of some or all features of functional section 30 is typically performed at imaging and control system 12.

FIG. 2 is a schematic diagram of endoscopy system 10 of FIG. 1 comprising imaging and control system 12 and endoscope 14. FIG. 2 schematically illustrates components of imaging and control system 12 coupled to endoscope 14, which in the illustrated example comprises a duodenoscope. Imaging and control system 12 can comprise control unit 16, which can include or be coupled to image processing unit 42, treatment generator 44 and drive unit 46, as well as light source unit 22, input unit 20 and output unit 18. Coupler section 36 can be connected to control unit 16 to connect to endoscope 14 to multiple features of control unit 16, such as image processing unit 42 and treatment generator 44. In examples, plug portion 48 of coupler section 36 can include leads 49 for connecting to wiring within socket 37 that can connect to light source unit 22, imaging processing unit 42 and treatment generator 44. In examples, port 40A can be used to insert another instrument or device, such as a daughter scope or auxiliary scope, into endoscope 14. Such instruments and devices can be independently connected to control unit 16 via cable 47 or can extend directly from fluid source 24 and suction pump 26 without coming from control unit 16. In examples, port 40B can be used to connect coupler section 36 to various inputs and outputs, such as video, air, light and electric. Control unit 16 can be configured to activate a camera to view target tissue distal of endoscope 14. Likewise, control unit 16 can be configured to activate light source unit 22 to direct light into endoscope 14 or other devices extending therefrom. Light source unit 22 can comprise a light generator, such as a xenon bulb or a light emitting diode. In example, light source unit 22 can include multiple light generators to generate light with different properties, such as different color.

Image processing unit 42 and light source unit 22 can each interface with endoscope 14 (e.g., at functional section 30) by wired or wireless electrical connections. Imaging and control system 12 can accordingly illuminate an anatomical region, collect signals representing the anatomical region, process signals representing the anatomical region, and display images representing the anatomical region on output unit 18. Imaging and control system 12 can include light source unit 22 to illuminate the anatomical region using light of desired spectrum (e.g., broadband white light, narrow-band imaging using preferred electromagnetic wavelengths, and the like). Imaging and control system 12 can connect (e.g., via an endoscope connector or socket 37 (FIG. 1)) to endoscope 14 for signal transmission (e.g., light output from light source, video signals from imaging system in the distal end, diagnostic and sensor signals from a diagnostic device, and the like).

Fluid source 24 (FIG. 1) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as an activation energy for a biasing device or a pressure-applying device of the present disclosure. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

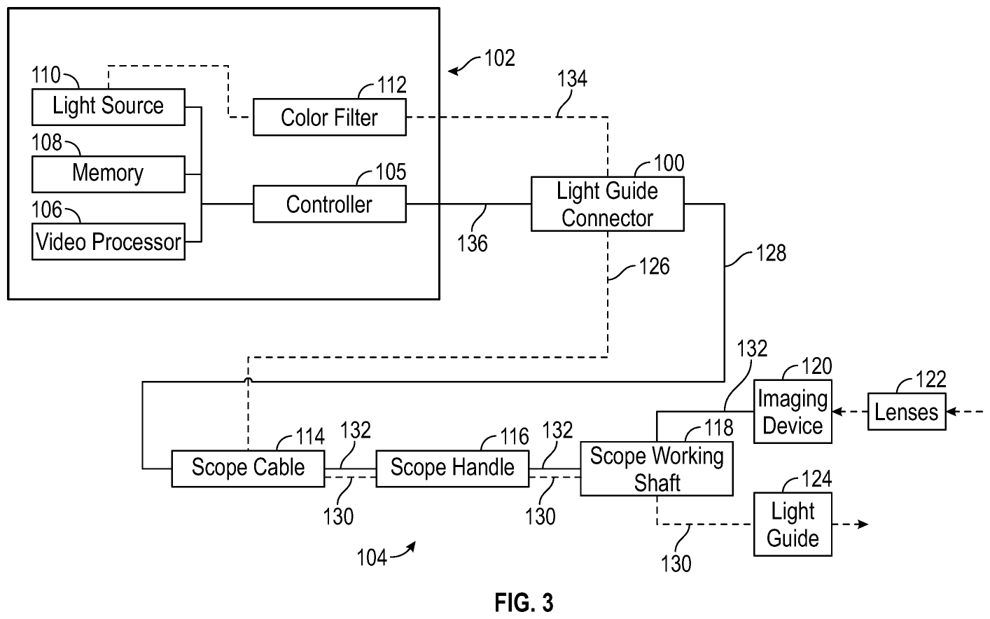

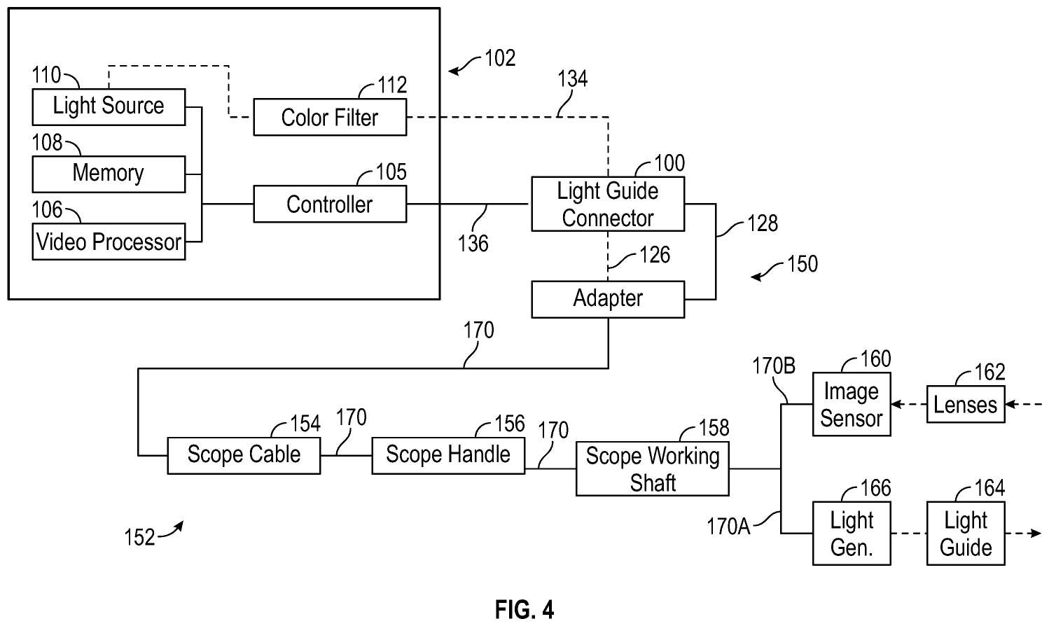

As mentioned, coupler section 36 can be used to connected endoscope 14 with imaging and control system 12. Coupler section 36 can be used to communicate various functions between endoscope 14 and imaging and control system 12. In examples, coupler section 36 can transmit communication signals, electronic signals, electrical signals, power signals, fluids including water and air, light waves and the like. Coupler section 36 can comprise a part of endoscope 14 and can be configured for particular configurations of imaging and control system 12. For example, coupler section 36 can be configured to transmit light generated by light source unit 22 to endoscope 14 using light conductor 39, as is discussed with reference to FIG. 3. With the present disclosure, a light processing adapter can be connected to imaging and control system 12 to couple to an endoscopes having built-in or on-board light generators. Such light processing adapters can convert light generated by light source unit 22 to electronic instructions for operating the on-board light generator to replicate the light generated by light source unit 22, as discussed with reference to FIG. 4.

FIG. 3 is a block diagram showing light guide connector 100 coupling imaging and control system 102 to endoscope 104. Imaging and control system 102 can comprise an instance of imaging and control system 12 of FIGS. 1 and 2. Imaging and control system 102 can comprise controller 105, video processor 106, memory 108, light source 110 and filter 112. In examples, controller 105 can comprise an instance of control unit 16 of FIG. 2, light source 110 can comprise an instance of light source unit 22 of FIG. 2, and video processor 106 can comprise an instance of image processing unit 42 of FIG. 2. Endoscope 104 can comprise scope cable 114, scope handle 116, scope working shaft 118, imaging device 120, lens 122 and light guide 124. In examples, scope cable 114 can comprise an instance of cable section 34 of FIG. 2, scope handle 116 can comprise an instance of handle section 32 of FIG. 2, and scope working shaft 118 can comprise an instance of insertion section 28 of FIG. 2. In examples, light guide connector 100 can comprise an instance of socket 37 of FIG. 1. As such, scope cable 114 can comprise a coupler similar to coupler section 36 of FIG. 1.

Light guide connector 100 can be used to convey electronic signals and light waves between imaging and control system 102 and endoscope 104. In FIG. 3, light waves can be indicated by dashed lines and wired signals can be indicated by solid lines. Light guide connector 100 can transmit electronic signals generated by imaging device 120 to imaging and control system 102 and control signals from controller 105 to endoscope 104. For example, control signals for operating various features of endoscope 104, such as ablation, suturing, RF signal generation, cryogenic features and the like, can be conveyed from controller 105 to endoscope 104. Furthermore, light waves from light source 110 can be conveyed to endoscope 104 via light guide connector 100.

Light guide connector 100 can include light conductor 126 and electric wiring 128. Endoscope 103 can include light conductor 130 and electric wiring 132. Imaging and control system 102 can include light conductor 134 and control wiring 136. Light conductor 126 of light guide connector 100 can connect light conductor 130 of endoscope 104 to light source 110 via light conductor 134, and electric wiring 128 of light guide connector 100 can connect electric wiring 132 of endoscope 104 to controller 105 via control wiring 136.

Endoscope 104 can control transmission of electronic imaging signals from imaging device 120 to imaging and control system 102. For example, light can enter lens 122 at endoscope 104. The light can be received by imaging device 120. In examples, imaging device 120 can comprise a charge-coupled device (CCD) or a solid state device such as a complementary metal oxide semiconductor (CMOS). Imaging device 120 can convert the light waves received from lens 122 to electronic signals. The electronic signals can be passed through scope working shaft 118, scope handle 116 and scope cable 114 via appropriate conductors of electric wiring 132 to electric wiring 128 of light guide connector 100. Electric wiring 128 of light guide connector 100 can include appropriate couplers for transmitting the electronic signal from imaging device 120 to imaging and control system 102 through control wiring 136. As such, video processor 106 can receive the electronic signals from imaging device 120 for displaying on a video monitor, such as output unit 18 of FIG. 1, after appropriate processing and the like. Memory 108 can include various red, green and blue image memories for processing signals generated by imaging device 120.

In addition to light signals and imaging signals, light guide connector 100 can relay other types of data, such as control signals for various functions of endoscope 104. In particular, control wiring 136, electric wiring 128 and electric wiring 132 can additionally be used to convey control signals for diagnostic and treatment functionality of endoscope 104. For example, a user can input setting for functionality of endoscope 104 in controller 105 using, for example, input unit 20 (FIG. 2). Controller 105 can then generate appropriate control signals for transmission to light guide connector 100. Electric wiring 128 can be configured to carry control signals with additional conductors or the same conductors that carry the imaging signals using, for example, leads 49 (FIG. 2).

Furthermore, though not illustrated in FIG. 3, light guide connector 100 can include appropriate tubing or piping to carry fluids, such as saline, irrigation fluid, air, insufflation gas and other gases and the like, to and from endoscope 104.

Light source 110 can control the intensity and type of light generated by imaging and control system 102. For example, light source 110 or features of imaging and control system 102, such as input unit 20 (FIG. 1), can include control features, such as buttons or knobs to start and stop generation of light waves, control the intensity of the light waves and the like. Imaging and control system 102 can additionally include control features for activating or deactivating different types of filters of filter 112, such as color filters and the like. Thus, a user of imaging and control system 102 can initiate settings for light to be transmitted to light guide 124 of endoscope 104 at imaging and control system 102. Typical user settings include: 1) on/off, 2) light intensity, and 3) light color. In examples, 1) the on/off setting can be a function of intensity (e.g., zero intensity equals off), 2) the intensity setting can be a function of current or electrical signal provided to light source 110, and 3) the color setting can be a function of which of filters 112 is applied to output of light source 110. Each of 1), 2) and 3) can be set by a user at controller 105 and can be indicated as a property of light waves emitted from light source 110. Light conductor 126 of light guide connector 100 can include appropriate couplers, conductors or pipes for transmitting light waves from light conductor 134 of light source 110 to light guide 124. As such, light waves from light source 110 can travel through filter 112, light conductor 134 of imaging and control system 12, light conductor 126 of light guide connector 100, light conductor 130 of endoscope 104 (including light conductor 39 of FIG. 2), and light guide 124, whereby the light waves can exit endoscope 14 to illuminate anatomy into which endoscope 104 is inserted.

Configured as such, light guide connector 100 can be configured to relay signals and light waves between imaging and control system 102 and endoscope 104 without modification. In examples, endoscope 104 can be specifically configured for operation with imaging and control system 102. For example, light guide 124 can be configured to transmit light waves generated by light source 110 without interruption or introducing any distortions, such as discolorations or intensity changes. Additionally, light guide connector 100 can provide an electronic communications pathway between imaging device 120 and video processor 106 and between controller 105 and functionality of endoscope 104. As such, light guide connector 100 does not include any capability for interpreting, analyzing or changing light signals, imaging signals and control signals. Furthermore, light guide connector 100 can be mechanically configured to couple to particular types of endoscope plugs, such as coupler section 36 of FIG. 1. Thus, other types of endoscopes not configured to receive the outputs of imaging and control system 12 or that are not mechanically configured to mate with socket 37 are not inter-operable or compatible with imaging and control system 12.

FIG. 4 is a block diagram showing adapter 150 of the present disclosure coupling imaging and control system 102 of FIGS. 1 and 2 to endoscope 152.

Imaging and control system 102 can comprise controller 105, video processor 106, memory 108, light source 110 and filter 112. Imaging and control system 102 can be configured similarly as described with reference to FIG. 3 to provide light output at light conductor 134 and to send and receive communication signals via control wiring 136.

Endoscope 152 can comprise scope cable 154, scope handle 156, scope working shaft 158, imaging device 160, lens 162, light guide 164 and light generator 166. Endoscope 152 can be configured similarly as endoscope 104 of FIG. 3, except that rather than endoscope 152 having light conductor 130 extending therethrough as in endoscope 104, endoscope 152 can include light generator 166. In examples, scope cable 154 can be configured similarly as cable section 34 of FIG. 2, scope handle 156 can be configured similarly as handle section 32 of FIG. 2, and scope working shaft 158 can be configured similarly as insertion section 28 of FIG. 2, with the inclusion of imaging device 160 instead of a light conductor proximal thereof. In examples, light guide connector 100 can comprise an instance of socket 37 of FIG. 1.

Adapter 150 can be used to convey information from light guide connector 100 to endoscope 152. Adapter 150 can be configured for insertion into socket 37 (FIG. 1) to receive light from light source unit 22 and control signals from control unit 16, as well as various air sources. Light guide connector 100 can be used to convey electronic signals and light waves between imaging and control system 102 and adapter 150. In FIG. 4, light waves can be indicated by dashed lines and wired signals can be indicated by solid lines. Adapter 150 can transmit electronic signals generated by imaging device 120 to light guide connector 100 for transmission to imaging and control system 102. Adapter 150 can additionally transmit electronic signals from controller 105 and light guide connector 100 to endoscope 152. Adapter 150 can receive light waves from light guide connector 100 generated by light source 110 and can convert such light waves into combined signal wiring 170 for transmission to light generator 166. Light generator 166 can comprise a light source configured to output light waves. In examples, light generator 166 can comprise a light emitting diode (LED). In additional examples, light generator 166 can be configured to generate light of different colors.

Adapter 150 can include combined signal wiring 170, which can extend through endoscope 152. Combined signal wiring 170 can branch into light signal wiring 170A for communicating with light generator 166 and imaging signal wiring 170B for communicating with imaging device 160. Imaging and control system 102 can include light conductor 134 and control wiring 136. Light conductor 126 and electric wiring 128 of light guide connector 100 can connect to adapter 150 and adapter 150 can transmit combined signal wiring 170 to light generator 166 and imaging device 160.

Endoscope 152 can control transmission of electronic imaging signals from imaging device 120 to imaging and control system 102. For example, light can enter lens 162 at endoscope 152. The light can be received by imaging device 160. In examples, imaging device 160 can comprise a charge-coupled device (CCD) or a solid state device such as a complementary metal oxide semiconductor (CMOS). Imaging device 160 can convert the light waves received from lens 162 to electronic signals. The electronic signals can be passed through scope working shaft 158, scope handle 156 and scope cable 154 via appropriate conductors of combined signal wiring 170 to electric wiring 128 of light guide connector 100. Electric wiring 128 of light guide connector 100 can include appropriate couplers for transmitting the electronic signal from adapter 150 to imaging and control system 102 through control wiring 136. As such, video processor 106 can receive the electronic signals from imaging device 160 for displaying on a video monitor, such as output unit 18 of FIG. 1, after appropriate filtering and the like.

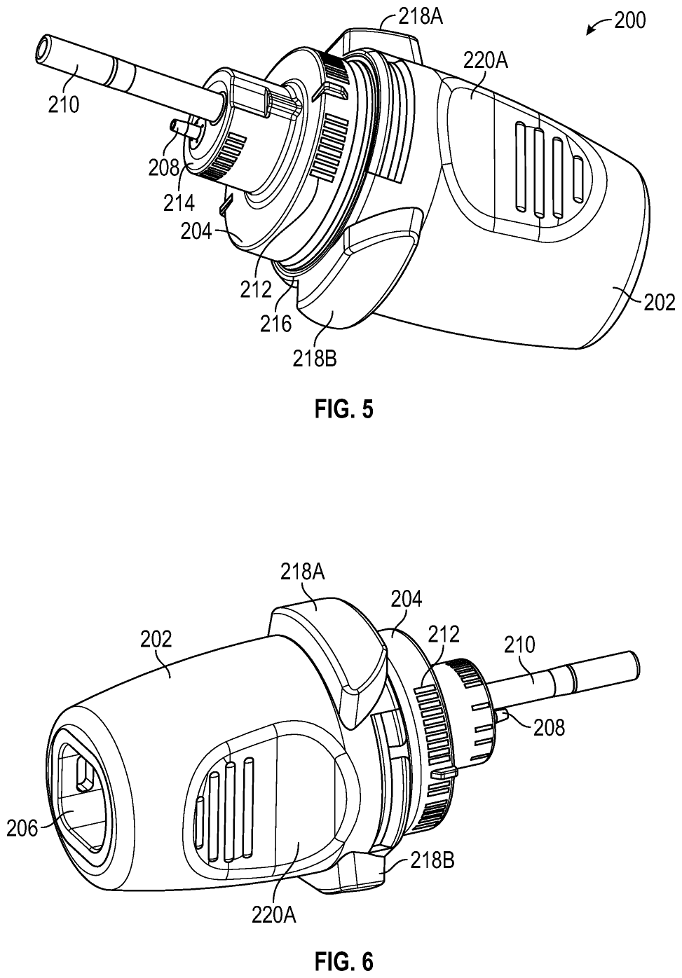

Light source 110 can control the intensity and type of light generated by imaging and control system 102, as explained above. For example, light source 110 or features of imaging and control system 102, such as input unit 20 (FIG. 1), can include control features, such as buttons or knobs to start and stop generation of light waves, control the intensity of the light waves and the like, to control 1) on/off of light source 110, 2) intensity of light from light source 11, and 3) color of light as determined by filters 112. Light conductor 126 of light guide connector 100 can include appropriate couplers, conductors or pipes for transmitting light waves from light conductor 134 of light source 110 to adapter 150. Adapter 150 can receive light waves from light conductor 134 and convert sensed properties, e.g., on/off, intensity and color, of the light waves into electronic control signal for light generator 166. Adapter 150 can include appropriate sensors and circuitry to convert light waves into electronic control signals, as is discussed with reference to FIGS. 5-15. As such, light waves from light source 110 can travel through filter 112, light conductor 126 of light guide connector 100 to adapter 150, followed by adapter 150 transmitting light generation signals along combined signal wiring 170 to light generator 166, which can thereafter output light to light guide 164, whereby the light waves can exit endoscope 152 to illuminate anatomy into which endoscope 152 is inserted. Thus, when an operator of imaging and control system 102 calls for light, e.g., instructs light source 110 to be on, light generator 166 can be commanded to generate light waves equivalent in intensity and color as light waves exiting light source 110.

Configured as such, adapter 150 can be configured to relay signals between imaging and control system 102 and endoscope 152 with translation, modification or interpolation. Endoscope 152 need not be specifically designed to operate with imaging and control system 102 and can include any type of light generator 166 and coupler section. In examples, endoscope 152 can be adapted for operation with imaging and control system 102 with the use of adapter 150. Adapter 150 can provide the appropriate mechanical interface between endoscope 152 and imaging and control system 102 and the appropriate translation of control inputs for 1), 2) and 3) entered at controller 105 to light generator 166. In addition to light signals, e.g., light waves, and imaging signal, e.g., electronic communication signals, adapter 150 can relay other types of data, e.g., control signals, as well as various fluids, such as water and air, between light guide connector 100 and endoscope 152. Adapter 150 can comprise a reusable part that is readily cleaned and sterilized, while endoscope 152 can be configured as a disposable scope that does not need to be cleaned or sanitized.

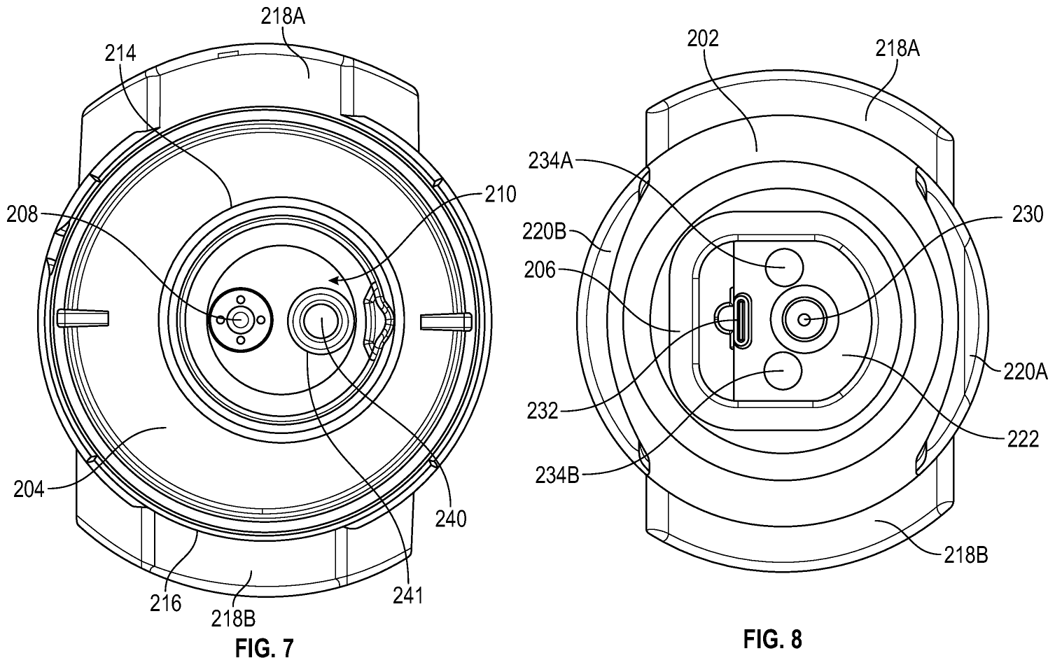

FIG. 5 is a rear perspective view of adapter 200 of the present disclosure showing main housing 202 and plug component 204. FIG. 6 is a front perspective view of adapter 200 of FIG. 5 showing socket component 206 for receiving an endoscope plug. Adapter 200 can comprise an instance of adapter 150 of FIG. 4. FIGS. 5 and 6 are discussed concurrently.

Plug component 204 can comprise air coupler 208, light conductor assembly 210 and electrical leads 212. Air coupler 208 and light conductor assembly 210 can extend from end face 214 of plug component 204. Electrical leads 212 can extend from shoulders or corners of plug component 204. Air coupler 208 and light conductor assembly 210 can be coupled to light guide connector 100 (FIG. 3) or directly to light source unit 22 (FIG. 1) or another component of imaging and control system 12. Plug component 204 can be inserted into receptacle 216 of main housing 202. Main housing 202 can comprise lugs 218A and 218B that can be coupled to a receptacle, e.g., socket 37 (FIG. 1) in light guide connector 100 or light source unit 22 via a twist-lock or push-pull operation. Main housing 202 can include other features such as pads 220A and 220B for providing ergonomic engagement with fingers of a user.

Adapter 200 can be configured to receive light waves at light conductor assembly 210, air at air coupler 208 and control signals at electrical leads 212 and 214. In examples, plug component 204 can be configured similarly as plug portion 48 of coupler section 36 (FIG. 2) and electrical leads 212 can operate similarly as lead 49. Thus, control signals generated by imaging and control system 12 (FIGS. 1 and 2) can be transmitted to adapter 200. Light conductor assembly 210 can be configured similarly as light conductor 39 (FIG. 2). Thus, light output by light source unit 22 can be conveyed to adapter 200.

Socket component 206 can include opening 222 to receive an endoscope plug, such as a plug connected to combined signal wiring 170 (FIG. 4). Opening 222 can include air coupler 230 (FIG. 8) and electrical coupler 232 (FIG. 8) for communicating with endoscope 152 (FIG. 4). As discussed with reference to FIGS. 7 and 8, adapter 200 can allow air and control signals to pass through main housing 202 and plug component 204 via fluid coupler 230 and electrical coupler 232.

FIG. 7 is a rear end view of adapter 200 of FIGS. 5 and 6 showing light conductor assembly 210 and air coupler 208. FIG. 8 is a front end view of adapter 200 of FIGS. 5 and 6 showing air coupler 230, electrical coupler 232, and alignment posts 234A and 234B. FIGS. 7 and 8 are discussed concurrently.

Plug component 204 can be inserted into socket 37 (FIG. 1) of light source unit 22. When inserted, electrical leads 212 and 214 can connect to electrical contacts within socket 37 to allow for transmission of electrical signals from imaging and control system 12, such as from control unit 16 and light source unit 22.

A plug for endoscope 152 (FIG. 4) can be inserted into opening 222. Opening 222 can have an irregular shape, such as a generally square shape with one side being rounded, to facilitate assembly with an endoscope plug in one orientation. Alignment posts 234A and 234B can be located in opening 222 to facilitate coupling with the endoscope plug. For example, alignment posts 234A and 234B can comprise cylindrical posts over which cylindrical sockets in the endoscope plug can slide to facilitate alignment. Additionally, alignment posts 234A and 234B can relive stress from being applied to fluid coupler 230 and electrical coupler 232. In additional examples, alignment posts 234A and 234B can be spring loaded to facilitate ejection of adapter 200. For example, alignment posts 234A and 234B can be biased to an extended position and then compressed when adapter 200 is connected to a control unit. As such, the force of the compressed springs can facilitate ejection of adapter 200 when pulled upon by an operator or user.

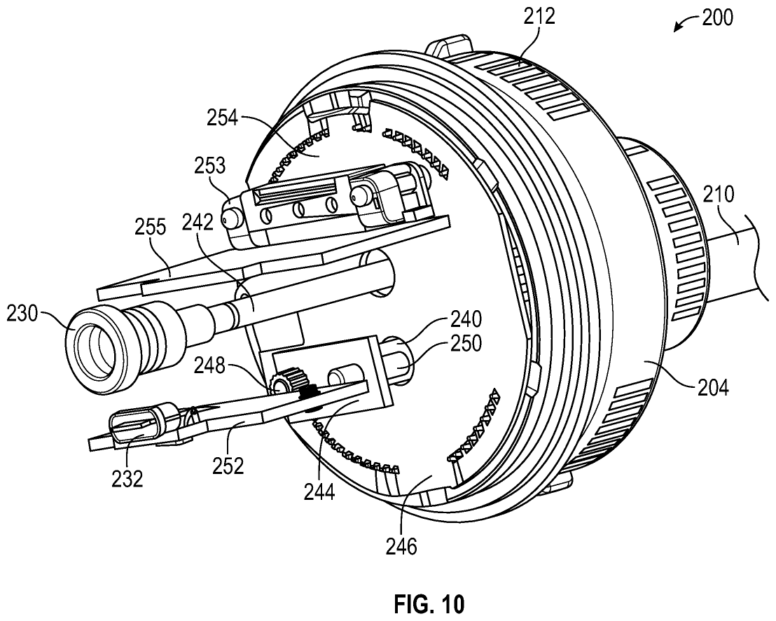

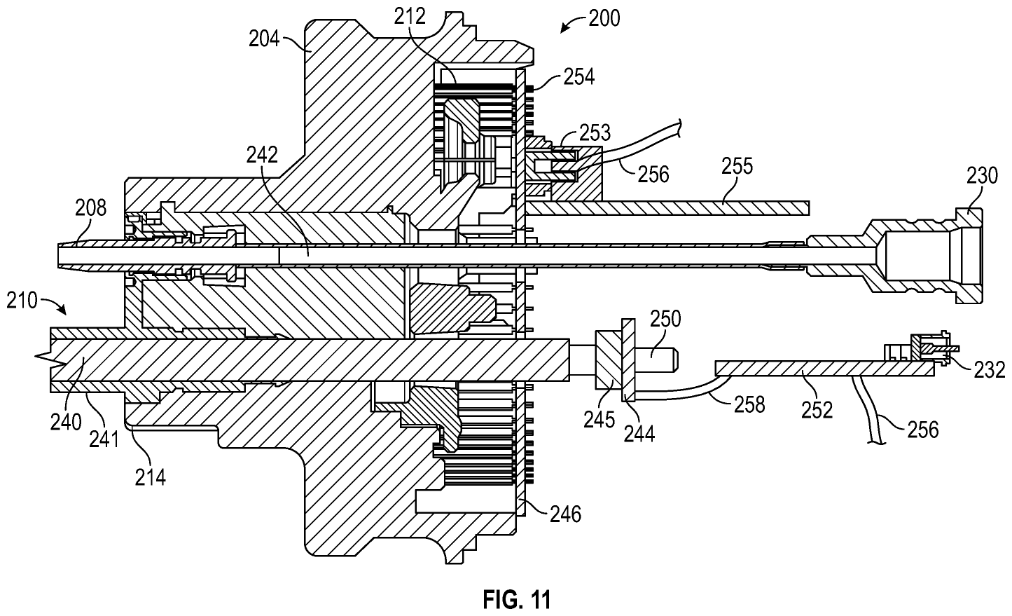

Air lines can be connected to air coupler 208 and air coupler 230. Air line 242 (FIGS. 10 and 11) can extend between air coupler 208 and air coupler 230 to allow air to pass through adapter 200. For example, air coupler 208 and air coupler 230 can be connected to air, carbon dioxide, saline, water and other fluid to perform various functions, including insufflation. In examples, air coupler 208 and air coupler 230 can comprise hose couplers or hose fittings with and without valves. In examples, adapter 200 can be configured to simply allow air to passthrough main housing 202 and plug component 204 without interference, adjustment or control. However, in some examples, adapter 200 can be configured to actively control air flow through main housing 202 and plug component 204 based on received electronic signals from control unit 16 (FIG. 1) or other sources, such as by including electronically controlled valves.

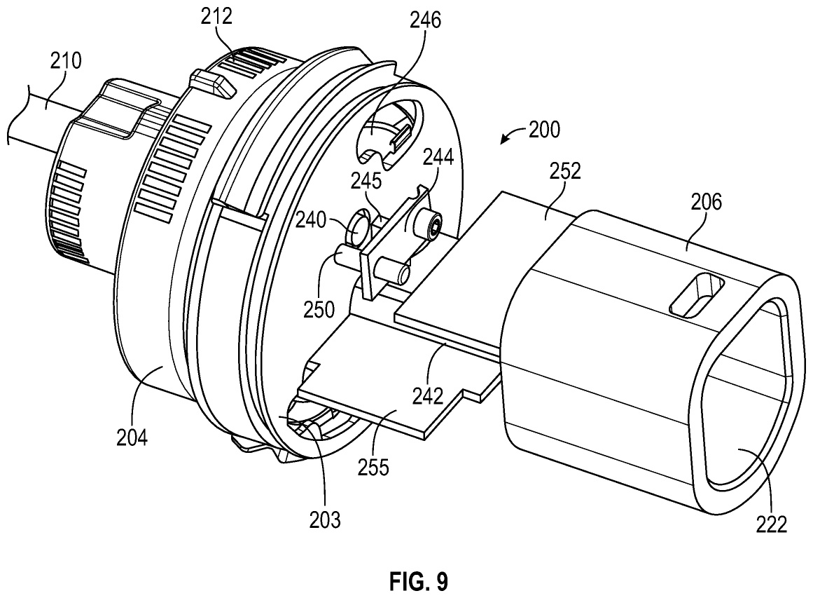

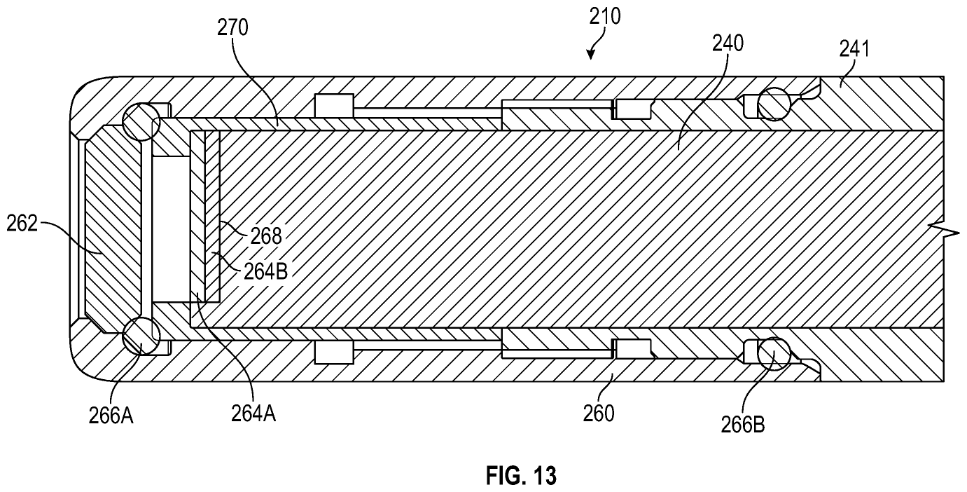

Light conductor assembly 210 can be configured to receive light waves from a light source. In particular, the end of light conductor assembly 210 can face the output of a light bulb or LED within light source unit 22. Light conductor assembly 210 can extend into plug component 204 and discharge the light waves onto sensor package 245 (FIG. 9). As discussed in greater detail below, electronics connected to sensor package 245 can translate the light waves into instructions for light generator 166 (FIG. 4) that can be transmitted through electrical coupler 232. In examples, light conductor assembly 210 can comprise light conductor 240 disposed in sheath 241. In examples, light conductor 240 can comprise a light pipe or a bundle of light fibers. For example, light conductor 39 (FIG. 2) of endoscope 14 can comprise a bundle of light fibers because light conductor 39 can comprise the proximal-most end of a bundle of light fibers extending through cable section 34 and insertion section 28. Thus, it is desirable to produce light conductor 39 from a plurality of light fibers to facilitate flexibility. However, light conductor 240 can comprise a light pipe, which can comprise a single piece light conductor having a diameter much larger than individual light fibers. As such, light conductor 240 can be rigid and more robust, such as by being more resistant to heat. Further description of light conductor assembly 210 is provided with reference to FIG. 13.

FIG. 9 is a front perspective view of adapter 200 of FIGS. 5 and 6 with main housing 202 removed to show support bracket 203 connected to plug component 204 and socket component 206. FIG. 9 additionally shows sensor package 245. FIG. 10 is a front perspective view of adapter 200 of FIG. 9 with support bracket 203 and socket component 206 removed to show air coupler 230, electrical coupler 232, light conductor 240 and air line 242. FIGS. 9 and 10 are discussed concurrently.

Light conductor 240 can be connected to light conductor assembly 210 extending from plug component 204. Light conductor can direct light onto sensor package 245. Air line 242 can be connected to air coupler 230 and air coupler 208 (FIGS. 5 and 7). Light board 244 can be connected to control board 246 of plug component 204 via fastener 248 and post 250. Control board 246 can be connected to prongs 254 of electrical leads 212. Control board 246 can be connected to communication board 252 via connector 253, which can be mounted on board 255. Connector 253 can be connected to communication board 252 via wiring 256 (FIG. 11). Light board 244 can be connected to communication board 252 via wiring 258 (FIG. 11).

Communication board 252 can be connected to electrical coupler 232 for transmitting control and light generation signals to endoscope 152 (FIG. 4). As such, when plug component 204 is inserted into socket 37 (FIG. 1), electrical leads 212 can be placed in communication with endoscope 152 through prongs 254, communication board 252 and electrical coupler 232. Likewise, when plug component 204 is inserted into socket 37, air line 242 can be placed in communication with endoscope 152 through air line 242, air coupler 208 and air coupler 230. Additionally, light conductor assembly 210 can be placed in alignment with sensor package 245.

FIG. 11 is a cross-sectional view through adapter 200 of FIG. 10 showing air line 242 through adapter 200 and sensor package 245 positioned proximate light conductor 240.

Air line 242 can comprise a conduit coupled to air coupler 208 and air coupler 230. In examples, air line 242 can comprise a rubber or plastic pipe or tube. Air line 242 can be connected to appropriate fittings on air coupler 208 and air coupler 230 to provide a leak-proof passage through adapter 200. For example, air coupler 208 and air coupler 230 can include barbed fittings over which air line 242 can fit. Air coupler 208 can comprise a male projection that can be fit into a mating female receptacle in socket 37 (FIG. 1). Air coupler 230 can comprise a female receptacle that can receive a mating male projection on a coupler of scope cable 154 (FIG. 4). Air coupler 208 can be rigidly supported by end face 214 of plug component 204 and air coupler 230 can be rigidly supported by socket component 206. Air line 242 can extend unsupported through adapter 200 between air coupler 208 and air coupler 230. As such, support bracket 203 control board 246 can include appropriate openings to allow for the extension of air line 242 therethrough.

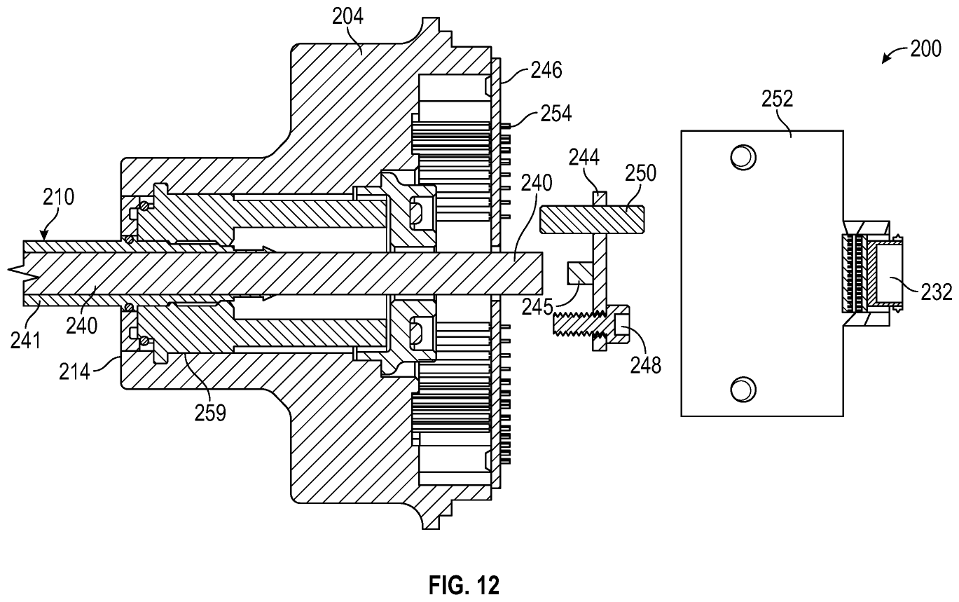

FIG. 12 is a cross-sectional view through adapter 200 of FIG. 10 showing light conductor 240 aimed at sensor package 245 mounted to light board 244 connected to electrical coupler 232. FIG. 12 is discussed with additional reference to FIG. 11.

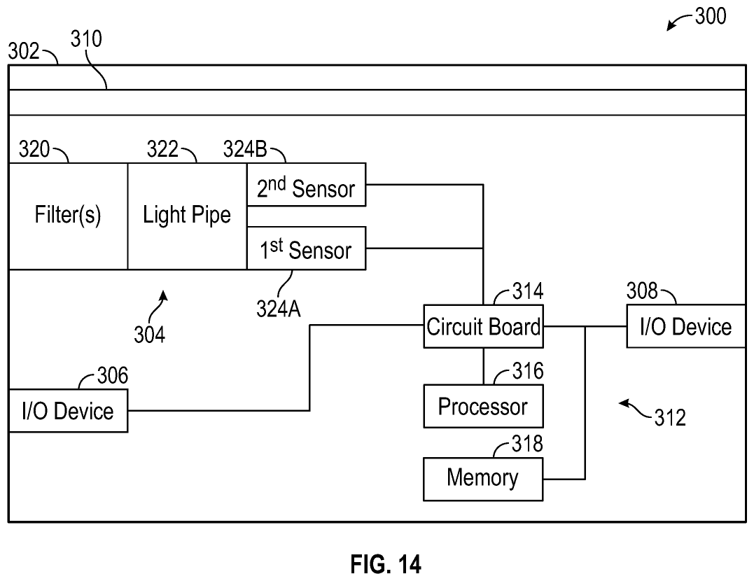

Light conductor assembly 210 can be attached to socket component 206. Specifically, sheath 241 can be inserted into receptacle 259 in end face 214 of socket component 206. Distal end of light conductor 240 can project through light board 244 and support bracket 203 to be located within main housing 202 proximate sensor package 245. Light board 244 can be mounted to support bracket 203 via fastener 248. Light board 244 can be placed in communication with communication board 252 via appropriate connections. In examples, wiring 258 can connect light board 244 and communication board 252. In other examples, light board 244 can be connected to control board 246. In examples, post 250 can be connected to support bracket 203 to provide alignment. Thus, output of sensor package 245 can be shared with other electrical components of adapter 200. As discussed with reference to FIG. 14, sensor package 245 can comprise one or more light sensors for interpreting various properties of light waves emanating from light conductor 240. Sensor package 245 or other appropriate electronics can convert output of the light sensors into instructions for operating light generator 66 (FIG. 4). The instructions for operating light generator 66 can be communicated to electrical coupler 232, along with other control signals from electrical leads 212. In examples, electrical coupler 232 can comprise an input/output device configured to send and receive electronic communications signals, as well as electrical power, e.g., current. In examples, electrical coupler 232 can comprise a Universal Serial Bus (USB) port, specifically a USB-C port. Thus, output from sensor package 245 and various prongs 254 from control unit 16 can be passed along to various components of endoscope 152 (FIG. 4).

FIG. 13 is a cross-sectional view through adapter 200 of FIG. 10 showing light conductor assembly 210. Light conductor assembly 210 can comprise light conductor 240, sheath 241, end cap 260, lens 262, first filter 264A, second filter 264B, first seal 266A and second seal 266B.

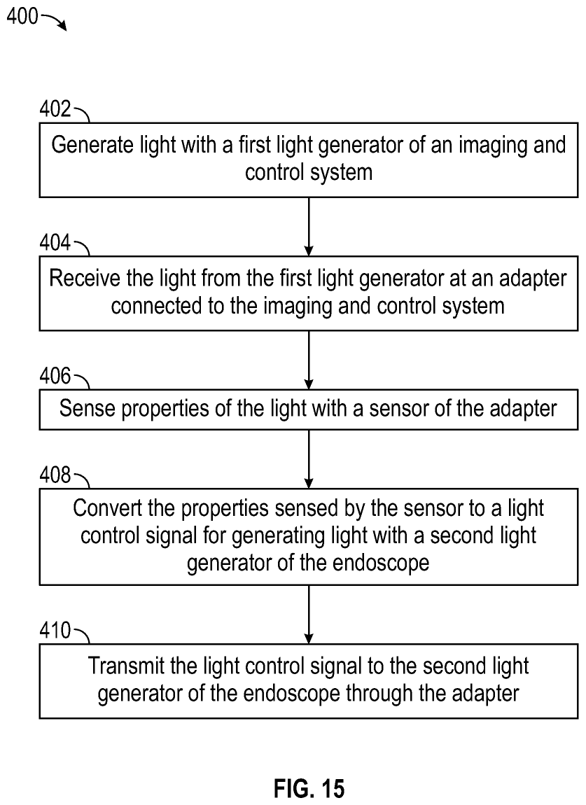

Proximal end of light conductor 240 can include face 268. Face 268 can be positioned to receive light waves exiting socket 37 (FIG. 1) that are produced by light source unit 22. Light conductor 240 can extend distally toward sensor package 245. Sheath 241 can surround a proximal portion of to facilitate assembly with plug component 204. Filters 264A and 264B can be placed proximate face 268 to receive light entering light conductor 240. Filters 264A and 264B can comprise polarizing films that are at an angle relative to each other to reduce the intensity of light transmitted to light conductor 240. In examples, the intensity of light entering light conductor 240 can be reduced as a safety feature to limit the temperature of light reaching sensor package 245. Furthermore, a very high light intensity can potentially electronically overwhelm the sensors within sensor package 245. Sensor package 245 can be configured to have memory with information stored therein about the amount of intensity reduction provided by filters 264A and 264B so that instructions for operating light generator 166 (FIG. 4) can be adjusted accordingly. Further discussion of the operation of sensor package 245 is provided with reference to FIGS. 14 and 15.

End cap 260 can be placed around filters 264A and 264B to secure filters 264A and 264B to face 268 of light conductor 240. End cap 260 can comprise lens 262 and fitting 270. Fitting 270 can comprise a holding device to retain filters 264A and 264B against light conductor 240. End cap 260 can subsequently be positioned over fitting 270 to hold filters 264A and 264B in place. Lens 262 can comprise a glass or crystal piece to allow light waves to pass therethrough without alteration. Lens 262 can protect filters 264A and 264B.

FIG. 14 is a block diagram illustrating light processing adapter 300 of the present disclosure. Light processing adapter 300 can comprise housing 302, light pipe assembly 304, first input/output (I/O) device 306, second input/output (I/O) device 308, air passage 310 and controller 312. Controller 312 can comprise circuit board 314, processor 316 and memory 318. Light pipe assembly 304 can comprise filters 320, light pipe 322, first sensor 324A and second sensor 324B.

Air passage 310 can be configured similarly as air coupler 208, air line 242 and air coupler 230. Air passage 310 can be configured as a pipe or tube to allow a fluid, such as air, gas, and water, to pass through adapter 300. Ends of air passage 310 can be provide with appropriate male or female fitting to connect to an imaging control system and an endoscope.

I/O device 306 can be configured as, or to communicate with, electrical leads 212, prongs 254 and control board 246. I/O device 306 can be configured to relay electronic communication signals into and out of adapter 300 for communication with an imaging and control system. I/O device 308 can be configured as electrical coupler 232. I/O device 306 can be configured to relay electronic communication signals into and out of adapter 300 for communication with a light-generating endoscope.

In examples, I/O device 306 and I/O device 308 can communicate using wireless communications signals, such as Bluetooth, WiFi, Zigbee, infrared (IR), near field communication (NFC), 3GPP or other technologies. In examples, I/O device 306 and I/O device 308 can comprise wired connections or can include ports for receiving wires for wired connections. In examples, I/O device 306 and I/O device 308 can communicate using one of more of the IEEE 802.15.6-2012 protocol, an MICS protocol and an MBANs protocol. In examples, I/O device 306 and I/O device 308 can comprise a port, such as a serial (e.g., Universal Serial Bus (USB) port, parallel port, or another wired or wireless (e.g., infrared (IR), near field communication (NFC), etc.) connection to communicate or control one or more features of an imaging and control system and endoscope.

Filters 320 can be configured as filters 264A and 264B. In examples, filters 320 can comprise absorptive filters that can absorb wavelengths of certain colors and that allow wavelengths of other colors to pass through. In examples, filters 320 can comprise interference filters that reflect wavelengths in certain spectral bands and that transmits wavelengths in other spectral bands. In examples, filters 320 can comprise a pair of polarizer filters rotationally offset to allows light waves of a specific polarization to pass through while blocking light waves of other polarizations.

Light pipe 322 can be configured as light conductor 240. Light pipe 322 can comprise a single-piece or monolithic component fabricated from optical acrylic or polycarbonate or other materials. In alternative example, light pipe 322 can be replaced by a bundle of optical fibers made of silica or plastic or other materials. Light pipe 322 can extend between filter 320 and sensors 324A and sensor 324B. As such, light exiting filters 320 can enter one end face of light pipe 322 and light exiting the opposite face of light pipe 322 can direct light waves onto sensor 324A and sensor 324B.

First sensor 324A and second sensor 324B can be configured as a portion of sensor package 245 (FIGS. 11 and 12). In examples, first sensor 324A can comprise a light intensity sensor. In examples, first sensor 324A can comprise a photodiode, a photoresistor, a phototransistor, and a photovoltaic light sensor. In examples, second sensor 324B can comprise a color sensor. In examples, second sensor 324B can comprise a light-to-photocurrent conversion sensor, a light-to-analog-voltage conversion sensor, and a light-to-digital conversion sensor.

Circuit board 314 can comprise a structural component for electrically and structurally coupling electrical components of adapter 300. For example, circuit board 314 can comprise a silicon wafer or a chip onto which electrical couplings are attached for electronic coupling of processor 316, memory 318, sensor 324A and sensor 324B and the like. Circuit board 314 as connected to processor 316, memory 318 and sensors 324A and 324B can operate as a converter for converting light waves into electronic signals as described herein.

Processor 316 can comprise an integrated circuit that controls operation of components of adapter 300, such as I/O devices 306 and 308, sensors 324A and 324B and memory 318. Processor 316 can execute instructions stored in memory 318 to operate components of adapter 300, such as sensors 324A and 324B. In examples, a processor and memory are not needed and adapter 300 can operate as a simple integrated circuit whereby output of sensors 324A and 324B can be directly transmitted by I/O devices 306 and 308.

Memory 318 can comprise any suitable storage device, such as non-volatile computer-readable memory, magnetic memory, flash memory, volatile memory, programmable read-only memory and the like. Memory 318 can include instructions stored therein for processor 316 to control operation of adapter 300. For example, memory 318 can include instructions for operating I/O devices 306 and 308 and sensors 324A and 324B. Memory 318 can additionally include reference data for comparing to data from sensors 324A and 324B, such as lookup tables for correlating light intensity sensed to a power input to light generator 166 (FIG. 4) and other information, that can be used to convert a light wave of a particular intensity and color into one or more electronic signals for generating light of the same or approximately same intensity and color. In an example, memory 318 can include a lookup table having light intensity from zero to the maximum output of light source 110 (FIG. 4) that is correlated to current input to light generator 166 (FIG. 4) from zero to a maximum input to light generator 166.

In examples, memory 318 can include instructions for scaling light signals generated by light generator 166 based on the effects of filters 320. For example, memory 318 can include an appropriate scaling factor to apply to the lookup tables discussed above. For example, processor can determine that filters 320 reduce the output of light source 110 by fifty percent such that the output of sensors 324A and 324B can be increased fifty percent before consulting the appropriate current to generate for operating light generator 166.

In additional example, memory 318 can include instructions to allow processor 316 to perform compensation for light source 110. For example, it is known that various light sources, such as xenon bulbs, dim, e.g., emit less light than desired, over time. Thus, an imaging and control system calling for a particular light intensity output may result in a light source outputting light having, for example, ninety-five percent of the called for intensity. Light processing adapters of the present disclosure can be configured to compensate for such dimming. In examples, imaging and control system 102 can be configured to provide 0% and 100% light intensity outputs for light source 110 at start-up. Light processing adapter 300 can have stored in memory 318 appropriate, e.g., intended undimmed output, 0% and 100% intensity outputs for particular models of imaging and control system 102. Thus, processor 316 can determine that light source 110 is only outputting 95% of the requested output from imaging and control system 102 and can appropriately upscale the output of light generator 166 such that the output of light generator 166 matches the called for light intensity at imaging and control system 102 even though light source 110 is not providing the called for light intensity.

FIG. 15 is a block diagram illustrating operations of method 400 for converting light generated by imaging and control system 12 into light control signals for light generator 166 of endoscope 152.

At operation 402, light can be generated with a first light generator of the imaging and control system. For example, light can be generated with light source 110 of imaging and control system 102 (FIG. 4). A user can input on/off, intensity and color settings at a user interface. For example, a user can utilize output unit 18 and input unit 20 (FIG. 1) to enter on/off, intensity and color settings for light source 110.

At operation 404, light from the first light generator, e.g., light source 110, can be received at adapter 300 connected to imaging and control system 102 (FIG. 4). Light waves from light source 110 can enter light conductor assembly 210 of adapter 300. Light conductor assembly 210 can be positioned opposite a light bulb or light emitting diode within light source 110 when adapter 300 is inserted into socket 37 (FIG. 1).

At operation 406, properties of light can be sensed with a sensor at the adapter. For example, first sensor 324A (FIG. 14) can be used to sense the intensity of light from light source 110. Additionally, in examples, second sensor 324B (FIG. 14) can be used to sense the color of light from light source 110. Light waves can exit light conductor assembly 210 and can be incident on first sensor 324A and second sensor 324B of sensor package 245. The light waves can energize appropriate elements of first sensor 324A and second sensor 324B to cause the generation of an electrical signal.

At operation 408, the light properties sensed by the sensors can be converted into a control signal for generating light with a second light generator of the endoscope. For example, light intensity sensed by first sensor 324A can be converted into instructions for generating light with light generator 166 (FIG. 4) of endoscope 152 (FIG. 4) at the same intensity. Additionally, in examples, light color sensed by second sensor 324B can be converted into instructions for generating light with light generator 166 of the same color.

In examples, processor 316 can receive signals from first sensor 324A relating to the intensity of light from light source 110. Light intensity from light source 110 can have a linear relationship to current input to light source 110. As such, current output from first sensor 324A can be scaled by processor 316 as control signal for light generator 166. Processor 316 can consult a lookup table stored in memory 318 having values of output of first sensor 324A associated with values of current to be provided to light generator 166 to produce the equivalent intensity of light output by light source 110. Memory 318 can be provided with lookup tables for different combinations of light source 110 and light generator 166. In examples, processor 316 can receive a signal from imaging and control system 12 providing an identification of light source 110, e.g., manufacturer, light type, bulb type, color type, LED type, etc., as well as an identification signal from endoscope 152 (FIG. 4) providing an identification of light generator 166, e.g., manufacturer, light type, bulb type, color type, LET type, etc. As such, processor 316 can consult the lookup table having the proper information for converting light output of the determined imaging and control system to a control input to the determined light generator 166. Furthermore, as discussed herein, processor 316 can condition the output of sensors 324A and 324 to accommodate light intensity filter conducted within adapter 300 using filters 320, as well as to provide light intensity compensation for dimming of output of light source 110 that occurs from prolonged use.

At operation 410, the light control signal can be transmitted to the second light generator of the endoscope through the adapter. For example, the light control signal generated by processor 316 can be transmitted to light generator 166 of endoscope 152 via combined signal wiring 170 and light signal wiring 170A. Light generator 166 can generate light waves having an intensity based on the received output of adapter 300. Furthermore, light generator 166 can produce light waves of a color called for by adapter 300. Thus, light output of light generator 166 can match the output of light source 110 in intensity and color. Light generator 166 can then discharge light waves that can be shone upon tissue, such as with the use of light guide 164 (FIG. 4).

As discussed herein, the present disclosure is useful in providing light generation instructions to disposable endoscopes, or reusable endoscopes, having on-board light generation capabilities, such as an LED using light processing adapters. The light processing adapters of the present disclosure allow endoscopes having on-board LEDs to receive light generation instructions from imaging and control systems that are not configured to communicate with endoscope light generators. As discussed herein, the light processing adapters of the present disclosure allow for the translation and transmission of instructions entered into an imaging and control system to be conveyed to a light-generating endoscope through the very light waves generated at the imaging and control system via the use of light sensors within the adapters. As such, light generating endoscopes, such as disposable endoscopes, can be used with existing capital equipment, such a imaging and control systems.

### EXAMPLES

Example 1 is an adapter for an endoscope system, the adapter comprising: a housing; a light conducting element extending into the housing; a sensor disposed within the housing to receive light waves emitted from the light conducting element; a converter connected to the sensor to convert light waves into an electrical signal comprising instructions for generating light with a light generator of an endoscope; and an electrical coupler connected to the converter and accessible through the housing configured to convey the electrical signal out of the housing to the endoscope.

In Example 2, the subject matter of Example 1 optionally includes wherein: the sensor comprises a light intensity sensor; and the converter comprises a lookup table for correlating a sensed light intensity to a power setting for the light generator of the endoscope.

In Example 3, the subject matter of Example 2 optionally includes wherein the converter comprises: a processor; and a non-transitory computer readable storage medium having the lookup table stored therein.

In Example 4, the subject matter of any one or more of Examples 1-3 optionally include a color sensor disposed within the housing to receive light waves emitted from the light conducting element; and the converter comprises a lookup table for converting output of the color sensor to instructions for the light generator of the endoscope to generate light having color of the light waves emitted from the light conducting element.

In Example 5, the subject matter of any one or more of Examples 1˜4 optionally include a filter for reducing an intensity of light waves impinging the sensor from the light conducting element.

In Example 6, the subject matter of Example 5 optionally includes wherein the filter comprises a pair of polarizing lenses.

In Example 7, the subject matter of any one or more of Examples 1-6 optionally include wherein the housing comprises a plug portion, the plug portion comprising: a plug body configured to be inserted into a socket of an imaging and control system; and an outlet in the plug body for the light conducting element.

In Example 8, the subject matter of Example 7 optionally includes wherein: the plug body further comprises electrical leads for connecting to electrical contacts in the socket of the imaging and control system; and the electrical coupler is configured to convey output of the electrical contacts and the converter to a control cable of an endoscope.

In Example 9, the subject matter of any one or more of Examples 1-8 optionally include a fluid passage extending through the housing, the air passage having an inlet and an outlet accessible from the housing.

Example 10 is a surgical endoscope system comprising: an imaging and control system comprising a light source having a socket; an endoscope comprising: a shaft comprising a coupler at a proximal end portion and an imaging device at a distal end portion; a working channel extending at least partially through the shaft; and a light generator configured to emit light proximate the distal end portion; and an adapter configured to be connected to the socket, the adapter comprising: a light sensor configured to receive light waves from the light source when the adapter is connected to the socket; a socket configured to receive the coupler of the endoscope; and a converter configured to transform light intensity readings from the light sensor into instructions for operating the light generator.

In Example 11, the subject matter of Example 10 optionally includes wherein the adapter comprises: a light conducting element configured to receive light waves from the light source and transmit the light waves to the light sensor when the adapter is connected to the socket.

In Example 12, the subject matter of any one or more of Examples 10-11 optionally include wherein the converter comprises: a memory device comprising a lookup table correlating light intensity values for the light source of the imaging and control system with power input values for the light generator of the endoscope; and a processor configured to receive an output from the light sensor and generate a command signal for the light generator using values in the lookup table.

In Example 13, the subject matter of any one or more of Examples 10-12 optionally include wherein the adapter further comprises a light intensity filter configured to reduce intensity of the light before impinging the light sensor.

In Example 14, the subject matter of any one or more of Examples 10-13 optionally include wherein the converter is configured to generate instructions for the light generator that scales-up light intensity of light waves emitted from the light source to compensate for diminishment of a light generate of the light source due to use.

In Example 15, the subject matter of any one or more of Examples 10-14 optionally include wherein: the light sensor comprises a light color sensor; and the light generator of the endoscope is configured to emit light in multiple colors.

In Example 16, the subject matter of any one or more of Examples 10-15 optionally include wherein the adapter further comprises: electrical leads configured to connect to electrical contacts in the light source; and an air passage extending through the adapter configured to connect an air outlet on the socket of the imaging and control system with an air inlet on the coupler of the endoscope.

Example 17 is a method for communicating light control signals from an imaging and control system to an endoscope having light generating capabilities, the method comprising: generating light with a first light generator of the imaging and control system; receiving the light from the first light generator at an adapter connected to the imaging and control system; sensing an intensity of the light with a sensor of the adapter; converting the intensity sensed by the sensor to a light control signal for generating light with a second light generator of the endoscope; and transmitting the light control signal to the second light generator of the endoscope through the adapter; wherein the light control signal is configured to instruct the second light generator of the endoscope to generate light of equivalent intensity sensed by the sensor.

In Example 18, the subject matter of Example 17 optionally includes wherein sensing an intensity of the light with the sensor of the adapter further comprises: reducing an of intensity of the light using the adapter before sensing the intensity of the light; and proportionately scaling up the light control signal to the intensity of the light before being reduced.

In Example 19, the subject matter of any one or more of Examples 17-18 optionally include sensing color of the light with a color sensor of the adapter; and providing a color generating instruction to the second light generator of the endoscope.

In Example 20, the subject matter of any one or more of Examples 17-19 optionally include generating a control instruction for a treatment or diagnostic feature of the endoscope with the imaging and control system; transmitting the control instruction to the endoscope through the adapter; and transmitting an air from the imaging and control system to the endoscope through the adapter.

In Example 21, the subject matter of Example 20 optionally includes testing degradation of the first light generator of the imaging and control system; and compensating for degradation of the first light generator with the second light generator of the endoscope.

Each of these non-limiting examples can stand on its own, or can be combined in various permutations or combinations with one or more of the other examples.

### Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An adapter for an endoscope system, the adapter comprising:
a housing;
a sensor to receive light waves emitted from a light source that can be connected to the adapter; and
a converter connected to the sensor to convert a light intensity reading from the sensor into a signal comprising instructions for generating light with a light generator of an endoscope;
wherein the adapter is configured to output the signal to the endoscope.

2. The adapter of claim 1, wherein:
the sensor comprises a light intensity sensor; and
the converter comprises a lookup table for correlating a sensed light intensity to a power setting for the light generator of the endoscope.

3. The adapter of claim 2, wherein the converter comprises:
a processor; and
a non-transitory computer readable storage medium having the lookup table stored therein.

4. The adapter of claim 1, further comprising:
a color sensor disposed within the housing to receive light waves emitted from the light source; and
the converter comprises a lookup table for converting output of the color sensor to instructions for the light generator of the endoscope to generate light having color of the light waves emitted from the light source.

5. The adapter of claim 1, further comprising a filter for reducing an intensity of light waves impinging the sensor from the light source.

6. The adapter of claim 5, wherein the filter comprises a pair of polarizing lenses.

7. The adapter of claim 1, further comprising:
a plug body configured to be inserted into a socket of an imaging and control system;
a light conducting element extending into the plug body to receive the light waves from the light source; and
an outlet in the plug body for the light conducting element.

8. The adapter of claim 7, wherein:
the plug body further comprises electrical leads for connecting to electrical contacts in the socket of the imaging and control system.

9. The adapter of claim 8, further comprising an electrical coupler connected to the converter and accessible through the housing configured to convey the signal of the converter and output of the electrical contacts to a control cable of an endoscope.

10. The adapter of claim 1, further comprising a wireless communication device for communication the signal out of the adapter.

11. The adapter of claim 1, further comprising a fluid passage extending through the housing, the fluid passage having an inlet and an outlet accessible from the housing.

12. A surgical endoscope system comprising:
an imaging and control system comprising a light source having a first socket;
an endoscope comprising:
a shaft comprising a proximal end portion and an imaging device at a distal end portion;
a working channel extending at least partially through the shaft; and
a light generator configured to emit light proximate the distal end portion; and
an adapter configured to be connected to the first socket, the adapter comprising:
a light sensor configured to receive light waves from the light source when the adapter is connected to the first socket; and
a converter configured to transform light intensity readings from the light sensor into instructions for operating the light generator.

13. The surgical endoscope system of claim 12, wherein the adapter comprises:
a light conducting element configured to receive light waves from the light source and transmit the light waves to the light sensor when the adapter is connected to the first socket.

14. The surgical endoscope system of claim 12, wherein the converter comprises:
a memory device comprising a lookup table correlating light intensity values for the light source of the imaging and control system with power input values for the light generator of the endoscope; and
a processor configured to receive an output from the light sensor and generate a command signal for the light generator using values in the lookup table.

15. The surgical endoscope system of claim 12, wherein the adapter further comprises a light intensity filter configured to reduce intensity of the light before impinging the light sensor.

16. The surgical endoscope system of claim 12, wherein the converter is configured to generate instructions for the light generator that scales-up light intensity of light waves emitted from the light source to compensate for diminishment of a light generate of the light source due to use.

17. The surgical endoscope system of claim 12, wherein:
the light sensor comprises a light color sensor; and
the light generator of the endoscope is configured to emit light in multiple colors.

18. The surgical endoscope system of claim 12, wherein the adapter further comprises:
electrical leads configured to connect to electrical contacts in the first socket; and
a second socket configured to receive a coupler located at the proximal end portion of the shaft of the endoscope.

19. The surgical endoscope system of claim 12, wherein the adapter further comprises:
a wireless communication device for communication the instructions to the endoscope.

20. A method for communicating light control signals from an imaging and control system to an endoscope having light generating capabilities, the method comprising:
generating light with a first light generator of the imaging and control system;
receiving the light from the first light generator at an adapter connected to the imaging and control system;
sensing an intensity of the light with a sensor of the adapter;
converting the intensity sensed by the sensor to a light control signal for generating light with a second light generator of the endoscope; and
transmitting the light control signal to the second light generator of the endoscope;
wherein the light control signal is configured to instruct the second light generator of the endoscope to generate light of equivalent intensity sensed by the sensor.

21. The method of claim 20, wherein sensing an intensity of the light with the sensor of the adapter further comprises:
reducing an of intensity of the light using the adapter before sensing the intensity of the light; and
proportionately scaling up the light control signal to the intensity of the light before being reduced.

22. The method of claim 20, further comprising:
sensing color of the light with a color sensor of the adapter; and
providing a color generating instruction to the second light generator of the endoscope.

23. The method of claim 20, further comprising:
generating a control instruction for a treatment or diagnostic feature of the endoscope with the imaging and control system;
transmitting the control instruction to the endoscope via the adapter; and
transmitting an air from the imaging and control system to the endoscope through the adapter.

24. The method of claim 23, further comprising:
testing degradation of the first light generator of the imaging and control system; and
compensating for degradation of the first light generator with the second light generator of the endoscope.

25. The method of claim 20, further comprising wirelessly transmitting the light control signal to the endoscope.
